(19) United States
(12) Patent Application Publication
(10) Pub. No.: US 2005/0143777 A1
(43) Pub. Date: Jun. 30, 2005

(54) Method and system of treatment of heart failure using 4D imaging

(76) Inventor: Jasbir S. Sra, Pewaukee, WI (US)

Correspondence Address: JANSSON, SHUPE & MUNGER, LTD, 245 MAIN STREET, RACINE, WI, 53403, US

(21) Appl. No.: 11/016,231

(22) Filed: Dec. 17, 2004

Related U.S. Application Data
(60) Provisional application No. 60/531,296, filed on Dec. 19, 2003.
(60) Provisional application No. 60/531,293, filed on Dec. 19, 2003.
(60) Provisional application No. 60/531,294, filed on Dec. 19, 2003.

(51) Int. Cl. A61N 1/362

(52) U.S. Cl. 607/4

(57) ABSTRACT

A method is provided for treatment of heart failure having the steps of obtaining cardiac digital data from a medical imaging system utilizing an ECG gated protocol; generating a series of 3D images of a cardiac chamber and its surrounding structures, preferably the left ventricle and coronary sinus, from this cardiac digital data at select ECG trigger points that correspond to different phases of the cardiac cycle; registering these 3D images with an interventional system; acquiring ECG signals from the patient in real-time; transmitting these ECG signals to the interventional system; synchronizing the registered 3D images with trigger points on the transmitted ECG signals to generate a 4D image; visualizing this 4D image upon the interventional system in real-time; visualizing a pacing/defibrillation lead over the 4D image upon the interventional system; navigating the pacing/defibrillation lead utilizing the 4D image; and placing the pacing/defibrillation lead over the cardiac chamber at an appropriate site to treat the heart failure.

### RELATED APPLICATION

This application claims the benefit of U.S. Provisional Applications Nos. 60/531,296, 60/531,293 and 60/531,294, each filed on Dec. 19, 2003 and the contents of each are incorporated by reference herein in its entirety.

### FIELD OF THE INVENTION

This invention relates generally to methods and systems for treatment of heart failure using bi-ventricular pacing/defibrillation leads and, in particular, to methods and systems utilizing 3D digital images for cardiac interventional procedures in such treatment and for the planning of such procedures.

### BACKGROUND OF INVENTION

Despite considerable progress in the management of congestive heart failure (CHF), it remains a major health problem worldwide. It is estimated that there are 6-7 million people with CHF in the United States and Europe and approximately 1 million patients are diagnosed with CHF every year.

Despite significant advances in the treatment of CHF using various pharmacological therapies, quality-of-life in patients with CHF is poor as they are frequently hospitalized and heart failure is a common cause of death. In addition, there is significant cost attached to this problem.

Normal electrical activation in the heart involves activation of the upper chambers called the atria followed by simultaneous activation of both the right and the left lower chambers called the ventricles by the left and right bundle branches. As patients with advanced CHF may have conduction system disease which may play a role in worsening cardiac function, pacing therapies have been introduced in an attempt to improve cardiac function. One frequently noted conduction abnormality is left bundle branch block (LBBB). In one study, 29% of patients with CHF had LBBB. Left bundle branch block delays left ventricular ejection due to delayed left ventricular activation as the electrical impulse has to travel from right to left side leading to sequential rather than simultaneous activation as mentioned before. In addition, different regions of the left ventricle may not contract in a coordinated fashion.

Cardiac resynchronization, also knows as bi-ventricular pacing, has shown beneficial results in patients with CHF and LBBB. During bi-ventricular pacing, both the right and left ventricle of the heart are paced simultaneously to improve heart pumping efficiency. It has also been shown recently that even some patients with no conduction system abnormalities such as the LBBB may also benefit from the bi-ventricular pacing. During bi-ventricular pacing, in addition to the standard right atrial and right ventricular lead used in currently available defibrillators or pacemakers, an additional lead is positioned into the coronary sinus. The lead is then advanced into one of the branches of the coronary sinus overlying the epicardial (outer) left ventricular surface. Once all the leads are in place, the right and left ventricular leads are paced simultaneously, thus achieving synchronization with atrial contraction.

There are, however, several problems with this approach. Initially, this type of approach is time-consuming for the physician. Placement of the left ventricular lead is limited to sites available that provide reasonable pacing and sensing parameters. Cannulating the coronary sinus can be challenging due to enlarged right atrium, rotation of the heart and presence of Tebesian valve (a valve close to the opening of the coronary sinus). Coronary sinus stenosis (occlusion) has also been reported in patients with prior coronary artery bypass surgery further complicating the problem. In most instances, problems with the placement of the coronary sinus lead are identified at the time of the interventional procedure. The procedure of coronary sinus lead placement is thus abandoned, the patient is brought back to the operating room and the left ventricular lead is positioned epicardially. During this procedure an incision is made on the lateral chest wall and the lead is placed on the outer side of the left ventricle.

Unfortunately, there are many problems with epicardial lead placement as well, some of which include but are not limited to:
I) Limited view of the posterolateral area of the left ventricle using the incision of the chest wall, also called minithoracotomy;
ii) The limited number of placement sites providing reasonable pacing and sensing parameters;
iii) Inability to identify the most appropriate location and placement of the lead at the most appropriate site;
iv) Potential risk of damaging the coronary arteries and venous system; and
v) Difficulty in identifying the ideal pacing site as a result of one or more of the above limitations.

Segmentation of various body organs can be performed from a radiological scan such as that performed by a computer tomography (CT) or magnetic resonance imaging (MRI) system, thereby yielding an explicit geometric description of those organs. Cardiac CT or other imaging techniques can be used to create a roadmap of coronary sinus and left ventricular anatomy such that appropriate sites can be identified for the placement of a left ventricular pacing lead for bi-ventricular pacing either at the most appropriate branch of the coronary sinus or on the left ventricular wall epicardially (from outside). CT or MRI can also identify areas devoid of blood vessels and nerves as well as scar tissue. These modalities can also be used to determine the asymmetric contraction of the ventricles and identify different regions of the ventricles not contracting in a coordinated fashion. The presence of scarring from previous heart attacks can make this uncoordinated contraction even worse. A method and system by which these anatomic structures can be registered with an interventional system and, with the aid of real-time visualization, leads can navigated in the 3D space and placed at the most appropriate site will make bi-ventricular pacing significantly safer and more effective.

A number of modalities exist for medical diagnostic imaging. The most common ones for delineating anatomy include CT, MRI and x-ray systems. CT systems are fast and accurate ways to delineate the anatomy of any organ. The ability to collect volumes of data at short acquisition times allows for 3-D reconstruction of images resulting in true depictions and more understandable anatomic images.

The role of CT in the management of cardiac rhythm problems has been, however, insignificant for several reasons which include motion artifacts in a beating structure such as the heart, and the inability to delineate the origin and propagation of electrical impulses. Use of cardiac gating allows acquisition of consecutive axial images from the same phase of a cardiac cycle. This will allow elimination of motion artifacts. Surface rendering techniques make it possible to view both endocardial (inside) and epicardial (outside) views of any chamber.

Although the 3D images of the different cardiac chambers could be created by the modalities mentioned before. These images even if they can be registered on an interventional system are still and do not replicate the motion of the heart real-time. It is thus not possible to assess the different aspects of the motion of the heart such as systole (contraction) or diastole (relaxation). This is critical if the pacing and defibrillation leads as in bi-ventricular pacing need to be navigated to the appropriate sites for successful results during the intervention procedure an to avoid complications such as perforation of the heart during the procedure as the exact orientation and location of the catheter or the pacing lead over the heart muscle is not possible in a still image.

The drawbacks discussed above and deficiencies of the prior art are overcome with a method and system of 4D imaging where the reconstructed 3D images are seen in real-time over different phases of the cardiac cycle.

### SUMMARY OF THE INVENTION

One aspect of this invention provides a method for treatment of heart failure in a patient using 4D imaging. The method has the steps of (1) obtaining cardiac digital data from a medical imaging system utilizing an electrocardiogram (ECG) gated protocol; (2) generating a series of three-dimensional (3D) images of a cardiac chamber and its surrounding structures from this cardiac digital data, the data having been gated at select ECG trigger points that correspond with different phases of the cardiac cycle; (3) registering these 3D images with an interventional system; (4) acquiring ECG signals from the patient in real-time; (5) transmitting these ECG signals to the interventional system; (6) synchronizing the registered 3D images with certain corresponding trigger points on the transmitted ECG signals such that a 4D image covering the different phases of the cardiac cycle is generated; (7) visualizing this 4D image upon the interventional system in real-time; (8) visualizing a pacing/defibrillation lead over the 4D image also upon the interventional system; (9) navigating the pacing/defibrillation lead utilizing the 4D image; and then (10) placing the pacing/defibrillation lead over the cardiac chamber at a select location to treat the heart failure.

In a desirable embodiment, the medical imaging system is a computer tomography (CT) system. Also preferred is where the imaging system is a magnetic resonance imaging (MRI) system or one utilizing ultrasound. Most desirable is where the method also includes the step of visualizing the 4D image over a computer workstation of the interventional system.

One very preferred embodiment finds the 3D images are of the left ventricle and coronary sinus. More preferred is where the select location is substantially devoid of features such as coronary vessels, nerves and scar tissue that would make it inappropriate for pacing and the method includes the step of utilizing the registered 3D images to identify this select location on the cardiac chamber. Most preferred is where the step of generating 3D images from the cardiac digital data uses a protocol optimized for 3D imaging of the left ventricle and coronary sinus.

Certain exemplary embodiments are where the interventional system is a fluoroscopic system. Also highly desired are embodiments having the additional step of continuously updating and adjusting the synchronization of the registered 3D images with the trigger points on the transmitted ECG signals during an interventional procedure.

Another aspect of this invention finds a system for treating heart failure in a patient. This system has a medical imaging system for obtaining cardiac digital data utilizing an electrocardiogram (ECG) gated protocol; an image generation system for generating a series of three-dimensional (3D) images of a cardiac chamber and surrounding structures from the cardiac digital data at select ECG trigger points that correspond to different phases of the cardiac cycle; an ECG monitor for acquiring ECG signals from the patient in real-time and for transmitting these ECG signals to an interventional system; a workstation for registering the 3D images with the interventional system and for then synchronizing these registered 3D images with trigger points on the transmitted ECG signals so as to generate a 4D image that is visualized upon the interventional system in real-time; and a pacing/defibrillation lead for placement over the cardiac chamber at a select location, the lead being visualized upon the interventional system over the 4D image.

A preferred embodiment is where the medical imaging system is a computer tomography (CT) system. Also preferred is where the 3D images are of the left ventricle and coronary sinus. More preferred is where the select location is substantially devoid of features that would make it inappropriate for pacing such as coronary vessels, nerves and scar tissue and the method includes the step of utilizing the registered 3D images to identify a select location on the cardiac chamber. Highly preferred cases find that the image generation system generates 3D images from the cardiac digital data utilizing a protocol optimized for 3D imaging of the left ventricle and coronary sinus.

In certain desirable embodiments, the interventional system is a fluoroscopic system. Most desirable is where the workstation continuously updates and adjusts the synchronization of the registered 3D images with the trigger points on the transmitted ECG signals during an interventional procedure.

In another aspect of this invention, a method is provided for planning treatment of a patient's heart failure. This method includes the steps of (1) obtaining cardiac digital data from a medical imaging system utilizing an electrocardiogram (ECG) gated protocol; (2) generating a series of three-dimensional (3D) images of a cardiac chamber and its surrounding structures having diminished cardiac function from the cardiac digital data at select ECG trigger points corresponding with different phases of the cardiac cycle; (3) registering the 3D images with an interventional system; (4) acquiring ECG signals from the patient in real-time; (5) transmitting the ECG signals to the interventional system; (6) synchronizing the registered 3D images with trigger points on the transmitted ECG signals to generate a 4D image; and (7) visualizing the 4D image upon the interventional system in real-time.

Yet another aspect of this invention finds a system for planning treatment of heart failure. The system comprises a medical imaging system for obtaining cardiac digital data utilizing an electrocardiogram (ECG) gated protocol; an image generation system for generating a series of three-dimensional (3D) images of a cardiac chamber and its surrounding structures having diminished cardiac function from the cardiac digital data at select ECG trigger points that correspond to different phases of the cardiac cycle; an ECG monitor for acquiring ECG signals from the patient in real-time and for transmitting these ECG signals to an interventional system; and a workstation for registering the 3D images with the interventional system and for synchronizing the registered 3D images with trigger points on the transmitted ECG signals to generate a 4D image that is visualized upon the interventional system in real-time.

### BRIEF DESCRIPTION OF THE DRAWINGS

FIG. 1 is a schematic overview of a system for treatment of heart failure in accordance with this invention.

FIG. 2 illustrates visualization of a standard pacing lead in real-time over a 3D image of the left ventricle registered upon an interventional system.

FIG. 3 is a flow diagram of a method for treatment of heart failure in accordance with this invention.

FIG. 4 is an example of 3D images of the left ventricle that are depicted as being synchronized to the systole (contraction) and diastole (relaxation) phases of the cardiac cycle.

### DETAILED DESCRIPTION OF PREFERRED EMBODIMENTS

The drawings illustrate embodiments of a system and method for treating heart failure in a patient using 4D imaging in accordance with this invention. The embodiments shown enable an electrophysiologist, cardiologist and/or surgeon to plan in advance and to later perform an interventional procedure such as bi-ventricular pacing in a manner that makes the procedure simpler and more efficacious while decreasing the risk of complications.

Using imaging systems known in the art, 3D images are obtained of a cardiac chamber such as the left ventricle and the adjacent coronary sinus. These images include detailed 3D models of the left ventricle and endocardial views (i.e., navigator or views from the inside) of the coronary sinus. These images are then registered and synchronized with real-time cardiac motion on an interventional system such as a fluoroscopic system to generate a 4D image. In this manner, detailed 3D images acquired at different phases of the cardiac cycle prior to an interventional procedure constitute displacement profiles of the cardiac chamber that can be visualized sequentially in real-time during the procedure.

In addition, a pacing/defibrillation lead may be seen over these images so that the practitioner can navigate the lead to strategic locations over the left ventricle in a manner where the orientation and location of the lead is better understood to avoid complications such as perforation of the heart during the procedure.

Although the embodiments illustrated are described in the context of a CT imaging system, it will be appreciated that other imaging systems known in the art, such as MRI and ultrasound, are also contemplated with regard to obtaining cardiac digital data for generating 3D images of the heart. Similarly, although the interventional system is described in the context of fluoroscopy and an associated computer work station, other interventional systems are also contemplated. In addition to viewing the left ventricle, the anatomy of other cardiac chambers can also be imaged, registered and visualized.

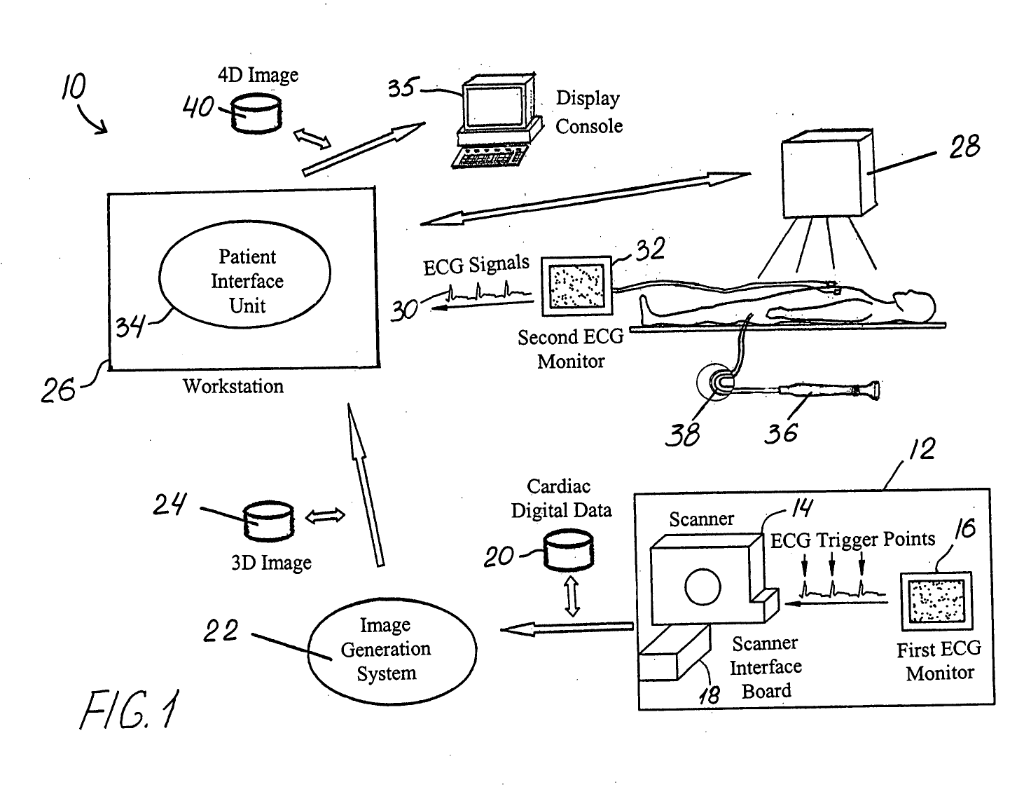

There is shown in FIG. 1 an schematic overview of an exemplary system 10 for treatment of heart failure in a patient in accordance with this invention. System 10 includes CT imaging system 12 having a scanner 14 and a first ECG monitor 16 that outputs ECG trigger points corresponding with different phases of the cardiac cycle to scanner 14 through a scanner interface board 18 utilizing a ECG gated protocol. A suitable example of scanner interface board 18 is a Gantry interface board. Scanner 14 therefore utilizes ECG-gated acquisition to image the heart at different phases of the cardiac cycle such as when the heart is free of motion and its diastolic phase, as well as in multiple phases of systole and early diastole.

Scanner 14 outputs cardiac digital data 20, including ECG signal time-stamps associated with such data generated by the gating protocol, to image generation system 22. Image generation is performed using one or more optimized 3D protocols for automated image segmentation of the cardiac digital data for the left ventricle and such surrounding structures as the coronary sinus. A series of gated 3D images 24 corresponding to the selected ECG trigger points are thus generated having quantitative features of the left ventricle such as its contour, orientation and thickness as well as providing endocardial or “immersible” views of the coronary sinus. 3D images 24 may be in any one of several formats, including but not limited to: a wire mess geometric model, a set of surface contours, a segmented volume of binary images, and a DICOM (Digital Imaging and Communications in Medicine) object using the radiation therapy DICOM object standard.

3D images 24 are exported from image generation system 22 and registered with workstation 26 of fluoroscopic system 28. ECG signals 30 are generated by second ECG monitor 32 and transmitted by ECG monitor 32 to workstation 26. ECG signals 30 contain data referable to an ECG being performed on the patient in real-time using ECG monitor 32 during the interventional procedure.

Workstation 26 includes patient interface unit 34 that places ECG signals 30 in communication with 3D images 24. Interface unit 34 is a processing unit that analyzes ECG signals 30 and synchronizes 3D images 24 with the real-time cardiac cycle of the patient by recognizing the ECG signal time-stamps on the images and matching them with the corresponding points on the real-time ECG. A zero time differential between these two values is calculated by workstation 26 to enhance synchronization. In this manner, 4D imaging 40 of the left ventricle is visualized on the interventional system at a display console 35.

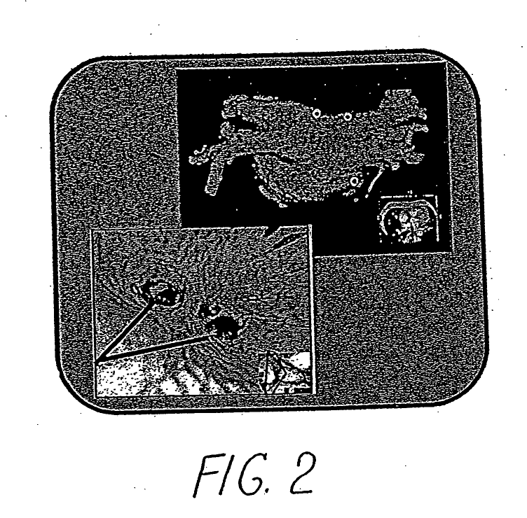

A detailed 3D model of the left ventricle registered upon an interventional system is shown in FIG. 2. A standard pacing lead is seen visualized in real-time over this image at a site selected to be the most appropriate for bi-ventricular pacing. The distance and orientation of the left ventricle and other strategic areas can be calculated in advance from such images. 3D images of this type are used to generate 4D imaging in accordance with this invention, thereby creating a roadmap for use during bi-ventricular pacing.

During the interventional procedure, a catheter apparatus 36 having a pacing/defibrillation lead 38 is delivered to the left ventricle typically by advancing the lead into a branch of the coronary sinus overlying the chamber's epicardial surface. Lead 38 is continuously localized on fluoroscopic system 28 whereby lead 38 is visualized over 4D image 40. Having lead 38 seen over 4D image 40 in real-time enables the practitioner to safely and accurately navigate lead 38 in real-time to the appropriate site over the left ventricle for the placement of lead 38 in the treatment of the patient's heart failure.

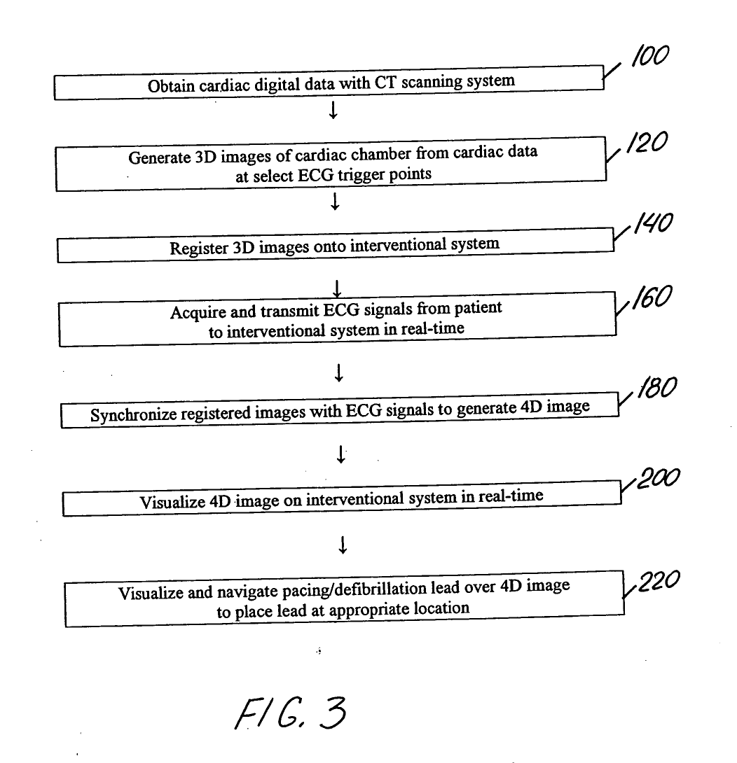

FIG. 3 illustrates a schematic overview of the method for treating heart failure using 4D imaging in accordance with this invention. As shown in step 100, the CT scanning system is used to obtain cardiac digital data. The CT imaging system is automated to acquire a continuous sequence of data of the patient's heart. A shorter scanning time using a faster scanner and synchronization of the CT scanning with a gated ECG signal of the patient at select trigger points reduces the motion artifacts in a beating organ like the heart and provides displacement profiles of the heart at different phases of the cardiac cycle. The ability to collect a volume of data in a short acquisition time allows reconstruction of cardiac images in more accurate geometric depictions, thereby making them easier to understand.

In step 120, the data-set acquired by the CT imaging system is segmented and a series of 3D images of the left ventricle and coronary sinus is generated using protocols optimized for those structures. The 3D images identify and visualize the desired views of the left ventricle at select points within the cardiac cycle.

As shown in step 140, the 3D images are then exported and registered with an interventional system such as one using fluoroscopy. The transfer of 3D images, including 3D model and navigator views, can occur in several formats such as DICOM format or object and geometric wire mesh model.

The registration method transforms the coordinates in the CT images into the coordinates in the fluoroscopic system. Information acquired by the CT scanning system will in this manner be integrated in real-time with imaging of the left atrium by the fluoroscopic system. Once these coordinates are locked in between the 3D images and the fluoroscopic views, the 3D models and navigator views can be seen from different perspectives on the fluoroscopic system.

At step 160, ECG signals are acquired from the patient at the time of the interventional procedure for performing bi-ventricular pacing. These signals are transmitted to the interventional system and brought into communication with the 3D images through a patient interface unit. In step 180, the interface unit analyzes the ECG signals received and synchronizes these signals with the gated 3D images to generate a 4D image. Several trigger points are recognized on both the real-time ECG and the ECG time-stamped 3D images and a zero time differential between these values is calculated.

As seen at step 200, this 4D image, comprising multiple views of the left ventricle and coronary sinus, can then be viewed sequentially in synchronization with the various phases of the cardiac cycle seen in real-time on the fluoroscopy system. Preferably, the synchronization of the 3D images with the real-time ECG signals is continuously updated and adjusted during the interventional procedure.

In addition, as shown at step 220, the invention further involves the location of a pacing/defibrillation lead over the fluoroscopic system and, in particular, over the registered 4D image of the left ventricle. The lead is then navigated to the appropriate site over the left ventricle in a less risky and efficient manner in treatment of the patient's heart failure.

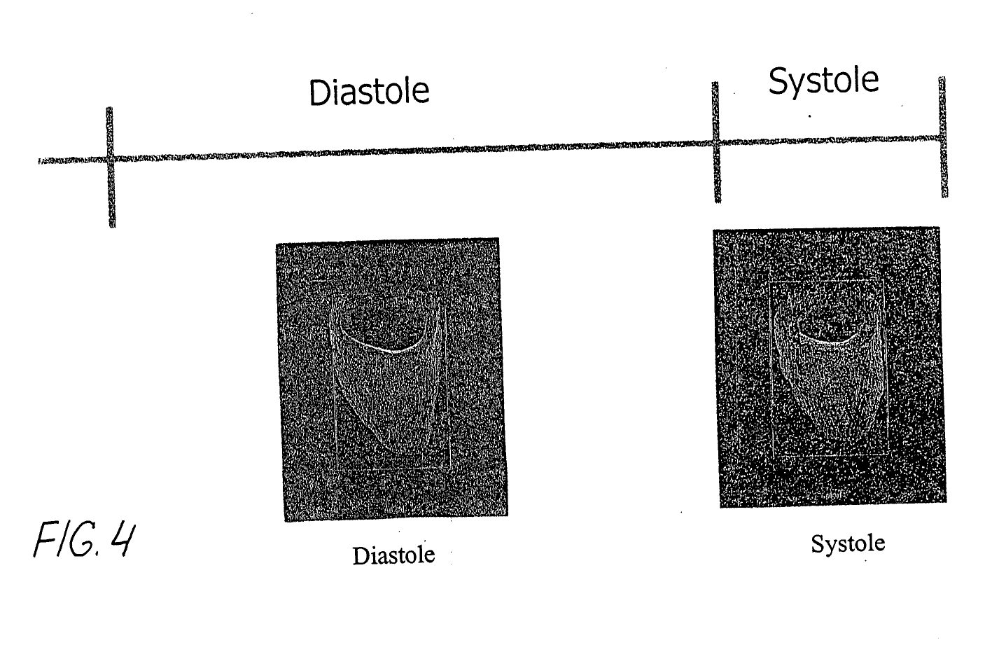

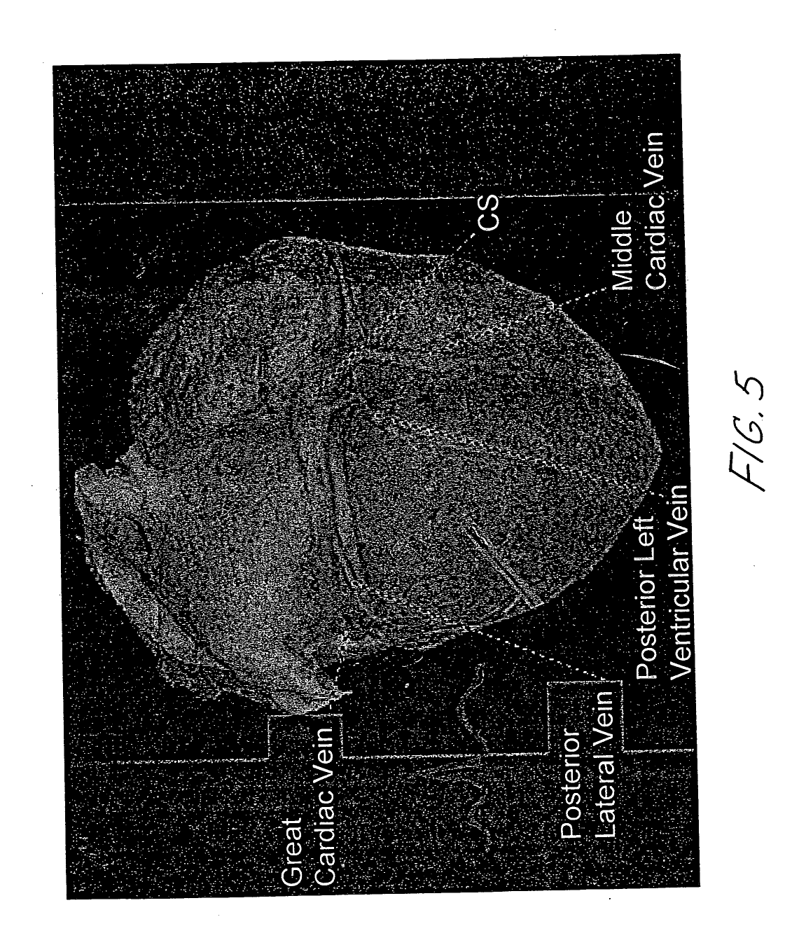

FIG. 4 is an example of 3D images depicting relaxation (diastole) and contraction (systole) of the left ventricle. The different displacement profiles are shown synchronized to a ECG signal where different trigger points are shown as small lines transecting the different phases of the cardiac cycle as shown by the horizontal line.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for treating heart failure in a patient using 4D imaging comprising:
obtaining cardiac digital data from a medical imaging system utilizing an electrocardiogram (ECG) gated protocol;
generating a series of three-dimensional (3D) images of a cardiac chamber and surrounding structures from the cardiac digital data at select ECG trigger points corresponding with different phases of the cardiac cycle;
registering the 3D images with an interventional system;
acquiring ECG signals from the patient in real-time;
transmitting the ECG signals to the interventional system;
synchronizing the registered 3D images with trigger points on the transmitted ECG signals to generate a 4D image;
visualizing the 4D image upon the interventional system in real-time;
visualizing a pacing/defibrillation lead over the 4D image upon the interventional system;
navigating the pacing/defibrillation lead utilizing the 4D image; and
placing the pacing/defibrillation lead over the cardiac chamber at a select location.

2. The method of claim 1 wherein the medical imaging system is a computer tomography (CT) system.

3. The method of claim 1 further comprising the step of visualizing the 4D image over a computer workstation of the interventional system.

4. The method of claim 1 wherein the 3D images are of the left ventricle and coronary sinus.

5. The method of claim 4 wherein the select location is substantially devoid of coronary vessels, nerves and scar tissue such that the select location is considered appropriate for pacing and further comprising the step of utilizing the registered 3D images to identify the select location.

6. The method of claim 5 wherein generating 3D images from the cardiac digital data comprises using a protocol optimized for 3D imaging of the left ventricle and coronary sinus.

7. The method of claim 1 wherein the interventional system is a fluoroscopic system.

8. The method of claim 1 further comprising the step of continuously updating and adjusting the synchronization of the registered 3D images with the trigger points on the transmitted ECG signals during an interventional procedure.

9. A system for treating heart failure in a patient using 4D imaging comprising:
a medical imaging system for obtaining cardiac digital data utilizing an electrocardiogram (ECG) gated protocol;
an image generation system for generating a series of three-dimensional (3D) images of a cardiac chamber and surrounding structures from the cardiac digital data at select ECG trigger points corresponding with different phases of the cardiac cycle;
an ECG monitor for acquiring ECG signals from the patient in real-time and for transmitting the ECG signals to an interventional system;
a workstation for registering the 3D images with the interventional system and for synchronizing the registered 3D images with trigger points on the transmitted ECG signals to generate a 4D image that is visualized upon the interventional system in real-time; and
a pacing/defibrillation lead for placement over the cardiac chamber at a select location, whereby the pacing/defibrillation lead is visualized over the 4D image upon the interventional system.

10. The system of claim 9 wherein the medical imaging system is a computer tomography (CT) system.

11. The system of claim 9 wherein the 3D images are of the left ventricle and coronary sinus.

12. The system of claim 11 wherein the select location is substantially devoid of coronary vessels, nerves and scar tissue such that the select location is considered appropriate for pacing and further comprising the step of utilizing the registered 3D images to identify the select location.

13. The system of claim 12 wherein the image generation system generates 3D images from the cardiac digital data utilizing a protocol optimized for 3D imaging of the left ventricle and coronary sinus.

14. The system of claim 9 wherein the interventional system is a fluoroscopic system.

15. The system of claim 9 wherein the workstation continuously updates and adjusts the synchronization of the registered 3D images with the trigger points on the transmitted ECG signals during an interventional procedure.

16. A method for planning treatment of heart failure in a patient using 4D imaging comprising:
obtaining cardiac digital data from a medical imaging system utilizing an electrocardiogram (ECG) gated protocol;
generating a series of three-dimensional (3D) images of a cardiac chamber and surrounding structures having diminished cardiac function from the cardiac digital data at select ECG trigger points corresponding with different phases of the cardiac cycle;
registering the 3D images with an interventional system;
acquiring ECG signals from the patient in real-time;
transmitting the ECG signals to the interventional system;
synchronizing the registered 3D images with trigger points on the transmitted ECG signals to generate a 4D image;
visualizing the 4D image upon the interventional system in real-time.

17. The method of claim 16 wherein the medical imaging system is a computer tomography (CT) system.

18. The method of claim 17 wherein generating 3D images from the cardiac digital data comprises using a protocol optimized for 3D imaging of the left ventricle and coronary sinus.

19. The method of claim 18 wherein the interventional system is a fluoroscopic system.

20. A system for planning treatment of heart failure in a patient using 4D imaging comprising:
a medical imaging system for obtaining cardiac digital data utilizing an electrocardiogram (ECG) gated protocol;
an image generation system for generating a series of three-dimensional (3D) images of a cardiac chamber and surrounding structures having diminished cardiac function from the cardiac digital data at select ECG trigger points corresponding with different phases of the cardiac cycle;
an ECG monitor for acquiring ECG signals from the patient in real-time and for transmitting the ECG signals to an interventional system;
a workstation for registering the 3D images with the interventional system and for synchronizing the registered 3D images with trigger points on the transmitted ECG signals to generate a 4D image that is visualized upon the interventional system in real-time.
